(19) United States
(12) Patent Application Publication
(10) Pub. No.: US 2004/0137037 A1
(43) Pub. Date: Jul. 15, 2004

(54) Plasma volume expanding formula

(75) Inventors: Kazunobu Okazaki, Matsumoto-shi (JP); Hideki Hayase, Saga-shi (JP); Tatsuya Doi, Kanzaki-gun (JP); Hiroshi Nose, Minamiazumi-gun (JP)

Correspondence Address: SUGHRUE MION, PLLC, 2100 PENNSYLVANIA AVENUE, N.W., SUITE 800, WASHINGTON, DC, 20037, US

(73) Assignee: PRESIDENT, SHINSHU UNIVERSITY AND OTSUKA PHARMACEUTICAL CO., LTD.

(21) Appl. No.: 10/634,125

(22) Filed: Aug. 5, 2003

(30) Foreign Application Priority Data
Sep. 27, 2002 (JP) 2002-283200

(51) Int. Cl. A61K 47/00

(52) U.S. Cl. 424/439

(57) ABSTRACT

The present invention provides a promoter for increasing plasma volume containing as an active ingredient a gel composition comprising the following components and having a pH in the range of 3 to 4: 3 to 8 wt. % of protein that does not coagulate at pH 3 to pH 4; 0.1 to 0.5 wt. % of calcium; 0.5 to 3 wt. % of acidulant; 4 to 20 wt. % of carbohydrate; 0 to 5 wt. % of fat; 0 to 0.5 wt. % of emulsifying agent; 0.1 to 1 wt. % of agar; and 65 to 90 wt. % of water.

### BACKGROUND OF THE INVENTION

(1) Technical Field

The present invention relates to a promoter for increasing plasma volume containing a specific gel composition as an active ingredient, and to a food containing the promoter.

(2) Prior Art

An increase in plasma volume is known to be beneficial in enhancing safety during physical exercise and athletic ability. For example, increased plasma volume increases the amount of skin blood flow. The increased skin blood flow increases heat emission, which suppresses the rise of core body temperature, thus being effective in improving heat tolerance and preventing heat stroke. Increased plasma volume also increases venous return, which reduces the heart rate, thereby enhancing athletic ability.

Endurance exercise increases plasma total protein content and circulatory plasma volume of young persons. In contrast, neither plasma total protein content nor circulatory plasma volume increased in elderly persons after 18 weeks of endurance exercise according to measurements done by the present inventors (results presented at the 56^{th} Annual Meeting of the Japanese Society of Physical Fitness and Sports Medicine (Sep. 20, 2001)). Zappe et al. have also published a similar report that plasma total protein content and circulatory plasma volume of young persons increased after repeated exercise, whereas neither increased in the elderly (Zappe Dh et al., “Age and regulation of fluid and electrolyte balance during repeated exercise sessions”, Am J Physiol. January 1996; 270(1 Pt 2): R71-9). Takamata et al. have also reported that young persons have increased circulatory plasma volume after repeated exercise, whereas elderly persons show no such increase (Takamata A et al., “Effect of an exercise-heat acclimation program on body fluid regulatory responses to dehydration in older men” Am. J. Physiol. October 1999; 277(4 Pt 2):R1041-R1050).

As shown above, the degree of change in plasma volume by repeated exercise varies between the young and the elderly. The mechanism by which plasma volume increases is not yet fully understood. In addition, means for effectively increasing plasma volume have not yet been fully researched or developed.

### BRIEF SUMMARY OF THE INVENTION

The main objective of the invention is to provide a means for effectively increasing plasma volume by revealing the mechanism by which plasma volume is increased.

To achieve the above object, the present inventors carried out intensive research and found that a pharmaceutical composition comprising a specific gel composition significantly increases plasma volume. The inventors conducted further intensive research and accomplished the present invention.

The present invention provides the following promoters for increasing plasma volume and food containing such a promoter.

1. A promoter for increasing plasma volume containing as an active ingredient a gel composition comprising the following components and having a pH in the range of 3 to 4:

| | | |
|---|---|---|
| Protein that does not coagulate at | 3-8 | wt. % |
| pH 3 to pH 4 | | |
| Calcium | 0.1-0.5 | wt. % |
| Acids | 0.5-3 | wt. % |
| Carbohydrate | 4-20 | wt. % |
| Fat | 0-5 | wt. % |
| Emulsifying agent | 0-0.5 | wt. % |
| Agar | 0.1-1 | wt. % |
| Water | 65-90 | wt. % |

2. The promoter according to item 1 wherein the gel composition further comprises 0.1 to 20 wt. % of at least one masking agent selected from the group consisting of fruit juice, fermented milk, hard-to-digest dextrin, hydrogenated resistant maltodextrin, nigerooligosaccharide and trehalose.

3. The promoter according to item 1 or 2 wherein the gel composition further comprises vitamin D in an amount of 0.1×10⁻⁶ to 10×10⁻⁶ wt. %.

4. A food containing the promoter of any of items 1 to 3.

### BRIEF DESCRIPTION OF THE DRAWINGS

FIG. 1 is a diagram showing an experimental scheme for administering the promoter for increasing plasma volume of the invention or a placebo shortly after exercise. The numerical value of the scale below the graph indicates the time of day. For example, 7 indicates 7:00 a.m. The numerals within the graph indicate the length of time (in minutes) needed for the experiment. In the graduations of the scale above the graph, C indicates just before exercise and the numerals indicate the lapse of time after exercise. The temperatures shown are ambient temperature of the laboratory. The arrows above the graph indicate the points in time when blood samples were collected. The arrow below the graph indicates the point in time when the promoter for increasing plasma volume of the invention or a placebo was administered.

FIG. 2 is a diagram showing the amount of change in plasma volume (PV), comparing the volume before exercise (baseline set as 0, point C in FIG. 1: the first blood collection) and the volume 23 hours after completion of the exercise (the eighth blood collection). In FIG. 2, the asterisk * indicates p<0.05 for the young versus the elderly comparison in the case of the promoter administration; *** indicates p<0.001 for the young versus the elderly comparison in the case of promoter administration and placebo administration; and p is the level of significance of misinterpreting the results. That is, p<0.05 and p<0.001 mean that the probability of missing the difference that in fact exists between the young and the elderly (obtaining false statistical results) is less than 5% and less than 0.1%, respectively.

FIG. 3 is a diagram showing the amount of change in plasma total protein content, comparing the content before exercise (baseline set as 0, point C in FIG. 1: the first blood collection) and the content 23 hours after completion of the exercise (the eighth blood collection). In FIG. 3, ***$ indicates p<0.001 for the young versus the elderly comparison in the case of promoter administration; ***# indicates p<0.001 for the young versus the elderly comparison in the case of placebo administration; and *** indicates p<0.001 for promoter administration versus placebo administration in both the young group and the elderly group.

FIG. 4 is a diagram showing the amount of change in plasma albumin content, comparing the content before exercise (baseline set as 0, point C in FIG. 1: the first blood collection) and the content 23 hours after completion of the exercise (the eighth blood collection). In FIG. 4, **$ indicates p<0.01 for the young versus the elderly comparison in the case of promoter administration; **# indicates p<0.01 for the young versus the elderly comparison in the case of placebo administration; and *** indicates p<0.001 for promoter administration versus placebo administration in both the young group and the elderly group.

### DETAILED DESCRIPTION OF THE INVENTION

The promoter for increasing plasma volume of the invention contains as an active ingredient a gel composition having specific components and a specific pH.

In this specification, “%” indicates “weight %”, unless otherwise specified.

Gel Composition

Protein

The gel composition contains protein as an essential ingredient. Protein is one of the three major nutrients, along with carbohydrate and fat. The proteins to be used are selected from proteins that do not coagulate in the pH of the gel composition of the invention, i.e., the pH range of 3 to 4.

Examples of useful proteins include Whey Protein Concentrate (WPC), Whey Protein Isolate (WPI), desalted whey, protein hydrolysates having a number average molecular weight of 500 to 10000, and the like. The protein of the invention may contain peptides and/or certain amino acids.

WPC and WPI are whey products obtained by subjecting liquid whey, a by-product produced during the production of milk products such as cheese and casein, to operations such as filtration, ion exchange, crystallization, precipitation and reverse osmosis or the like. Table 1 shows specific examples of WPC and WPI.

TABLE 1

| | WPC- | WPC- | WPC- | WPC- | WPC- | |
|---|---|---|---|---|---|---|
| | 34 | 50 | 60 | 75 | 80 | WPI |
| Protein | 34-36 | 50-52 | 60-62 | 75-78 | 80-82 | 90-92 |
| α- | 6.5 | 9.5 | 11 | 14 | 15 | 21 |
| Lactoalbumin | | | | | | |
| β- | 16 | 24 | 29 | 36 | 38 | 47 |
| Lactoalbumin | | | | | | |
| Serum albumin | 1.7 | 2.5 | 3.0 | 3.8 | 4.0 | 1.5 |
| Immuno- | 2.7 | 4.0 | 4.8 | 6.0 | 6.4 | 2.4 |
| globulin | | | | | | |
| Lactose | 48-52 | 33-37 | 25-30 | 10-15 | 4-8 | 0.5-1 |
| Fat | 3-4.5 | 5-6 | 1-7 | 4-9 | 4-8 | 0.5-1 |
| Ash | 6.5-8.0 | 4.5-5.5 | 4-6 | 4-6 | 3-4 | 2-3 |
| Water | 3.0-4.5 | 3.5-4.5 | 3-5 | 3-5 | 3.5-4.5 | 4.5 |
| Molecular | — | — | — | — | — | — |
| weight | | | | | | |
| Isoelectric | — | — | — | — | — | — |
| point | | | | | | |
| pH | 6-6.7 | 6-6.7 | 6-6.7 | 6-6.7 | 6-6.7 | 6.4 |

Desalted whey is obtained by pasteurizing whey at a low temperature and removing therefrom minerals by precipitation, filtration, dialysis or other separation techniques. Normally, it contains 79% carbohydrate, 2% fat, 13% protein and less than 7% ash.

When WPC, WPI or desalted whey is used, the proportion of protein in the gel composition of the invention is indicated by the amount of protein in the WPC, WPI or desalted whey.

Examples of protein hydrolysates having a number average molecular weight of about 500-10000 include proteins which do not coagulate at pH 3-4 or peptides obtained by hydrolyzing common proteins such as casein, gelatin, soybean protein and wheat protein using enzymes and acids to the molecular weight mentioned above.

They are usually composed of peptides wherein up to 100 amino acids are connected by peptide linkages. Amino acids may also be included in the protein hydrolysates.

Among the above proteins, WPC, WPI and protein hydrolysates having a number average molecular weight of 500 to 10000 are preferable. Hydrolysates of gelatin, soy protein and wheat protein are particularly preferable as protein hydrolysates having a number average molecular weight of 500 to 10000.

Proteins that do not coagulate at pH 3 to pH 4, as exemplified above, may be used as the protein of the invention singly or in combination of two or more.

The proportion of protein in the gel composition of the invention is preferably in the range of about 3 to about 8 wt. %, and more preferably about 4 to about 7 wt. %.

If necessary, the composition of the invention may contain proteins which coagulate at an acidic pH, in addition to protein materials which do not coagulate at pH 3 to pH 4.

Specific examples of proteins that coagulate in the acidic range include casein, soy protein, wheat protein and the like. Salts, fermented products, extracts or condensates of casein, soy protein or wheat protein are also usable. Whole milk powders, skim milk powders and the like are also usable. These proteins can be used singly or in combination of two or more.

The proportion of protein that coagulates in the acidic range in the gel composition is preferably less than 1 wt. %. Fermented protein products are preferable among the proteins that coagulate in the acidic range. Examples of fermented products include yogurt, cheese and the like.

The combined use of protein that does not coagulate at pH 3 to pH 4 and protein that coagulates in the acidic region can adjust the balance of protein content and improve taste.

Calcium

The gel composition of the invention contains calcium as an essential ingredient. Calcium is needed for the formation of bone and teeth and maintains normal blood calcium levels and the health of bones and teeth. In addition, calcium is an important nutritional component that smoothly activates the functions of the blood, heart and muscles.

Examples of raw materials to be incorporated as calcium-containing substances in the gel composition include natural calcium materials and synthetic calcium materials.

Examples of natural calcium materials include milk calcium, shell calcium, coral calcium, eggshell calcium, bone calcium, dolomite and the like.

Examples of synthetic calcium materials include calcium chloride, calcium lactate, calcium citrate, calcium carbonate, calcium pyrophosphate dihydrate, calcium gluconate and the like.

The proportion of calcium in the gel composition is preferably in the range of about 0.1 to about 0.5 wt. %, and more preferably about 0.1 to about 0.4 wt. %.

The proportion of calcium in the invention is indicated by the amount of calcium in the calcium raw material.

Acids

Acids are incorporated in the gel composition of the invention to adjust the pH to a range of 3 to 4, preferably 3.5 to 4.

It is preferable to use as acids at least two kinds of acidic components selected from the group consisting of citric acid, ascorbic acid, tartaric acid, succinic acid, malic acid, gluconic acid, phosphoric acid, phytic acid and lactic acid. Citric acid can be used in the form of trisodium citrate.

The proportion of acids in the gel composition of the invention is preferably in the range of about 0.5 to about 3 wt. %, and more preferably about 0.5 to about 2 wt. %.

The pH of the gel composition described in this specification is the pH value determined by the glass electrode method.

Carbohydrate

The gel composition of the invention contains carbohydrate as an essential ingredient. Carbohydrate is one of the three major nutrients and is stored as glycogen in the liver and muscles and consumed as an energy source when exercising, etc.

Specific examples of carbohydrate include monosaccharides such as glucose and fructose; disaccharides such as maltose and sucrose; sugar alcohols such as xylitol, sorbitol, glycerin and erythritol; polysaccharides such as dextrin and cyclodextrin; oligosaccharides such as fructooligosaccharide and galactooligosaccharide. These carbohydrates can be used singly or in combination of two or more. When two or more carbohydrates are used in combination, commercially available carbohydrate mixtures, for example, isomerized sugar or purified sucrose are usable.

Usable carbohydrates include those serving not only as nutrients but also as sweeteners, such as sucrose. Carbohydrates serving as sweeteners are preferably used, because they impart sweetness to the gel beverage composition.

The proportion of carbohydrate in the gel composition is preferably in the range of about 4 to about 20 wt. %, and more preferably about 5 to about 16 wt. %.

Fat

The gel composition of the invention may contain fats. Fats serve as substitute energy sources for carbohydrates during, for example, long-term physical exercise.

Examples of fats include long-chain fatty acid triglyceride (LCT), medium-chain fatty acid triglyceride (MCT) and the like as sources of essential fatty acids.

LCT is a triglyceride usually containing fatty acids having 11 or more carbon atoms and includes, for example, soybean oil, cottonseed oil, safflower oil, corn oil, rice oil, coconut oil, basil oil, sesame oil, linseed oil and like vegetable oils, sardine oil, cod liver oil and like fish oils, toad oil and the like.

MCT is a triglyceride usually containing fatty acids having 8 to 10 carbon atoms and includes, for example, caprylic acid, capric acid, lauric acid and the like. MCT is characterized by easy absorption, easy flammability and low accumulation.

LCT and MCT may be used singly or as a mixture of two LCTs, a mixture of two MCTs or a mixture of LCT and MCT.

The proportion of fats in the gel composition is about 0 to about 5 wt. %, and preferably about 0 to about 3 wt. %.

Emulsifying Agent

Fats are soluble in oil but sparingly soluble in water. Therefore, when fats are incorporated in a gel composition, an emulsifying agent for emulsifying the fat is incorporated to prepare the composition.

The emulsifying agent can be suitably selected from various emulsifying agent conventionally used in the field of beverage and food products. Considering that the composition of the invention is adjusted to the specified acidic pH, it is preferable that selection be made from emulsifying agent having acid resistance.

Typical examples of emulsifying agents are glycerol esters of fatty acids. Examples of useful glycerin fatty acid esters include various compounds known as emulsifying agents in the field of food products. For example, any of of highly purified monoglycerides, reactive monoglycerides, highly purified diglycerin mono fatty acid esters, and polyglycerine esters can be used.

Examples of usable commercially available products include “Sunsoft” (trademark, manufactured by TAIYO KAGAKU CO.,LTD.), “Emulsy” (trademark, manufactured by RIKEN VITAMIN CO.,LTD.) and “Ryoto” (trademark, manufactured by MITSUBISHI CHEMICAL CORPORATION).

In addition to glycerin fatty acid esters, other emulsifying agents used in the field of food products can be used in the present invention.

Examples of such emulsifying agents include phospholipids such as egg yolk lecithin, hydrogenated egg yolk lecithin, soybean lecithin and hydrogenated soybean lecithin; synthetic surfactants such as polyoxyethylene monooleate (e.g., commercially available product “Tween 80” manufactured by AMR), sucrose fatty acid esters, sorbitan fatty acid esters, propylene glycol fatty acid esters and the like.

The emulsifying agents may be used singly or in combination of two or more. The combined use of two or more emulsifying agents is usually preferable.

The proportion of emulsifying agents in the gel composition is preferably about 0 to about 0.5 wt. %, and more preferably about 0 to about 0.3 wt. %.

Agar

The gel composition of the invention contains agar as an essential ingredient.

Any agar which is extracted from red algae with hot water extraction, solidifying and drying the extract is useful. Red algae include Tengusa (Gelidium amansii), Ogonori (Gracilaria verrucosa), Obakusa (Pterocladia tenuis) and Itanikusa (Ahnfeltia plicata). Such agar includes agar strings, square agar, agar flakes, agar powders and the like.

Excellent texture is achieved by incorporating agar in the gel composition of the invention.

The proportion of agar in the gel composition is preferably about 0.1 to about 1 wt. %, more preferably about 0.2 to about 0.8 wt. %.

Other Gelling Agents or Thickening Agents

In addition to agar, if necessary, the composition of the invention may contain various substances conventionally used as gelling agents or thickening agents in the field of food products.

Examples of gelling agents include gellan gum, carrageenan, pectin, gelatin and the like. Examples of thickening agents include furcellaran, locust bean gum, guar gum, gum Arabic, xanthan gum and the like.

These gelling agents and thickening agents can be used singly or in combination of two or more. The combined use of gelling agents and thickening agents is particularly preferable.

Gelling agents and/or thickening agents exhibit an appropriate gelling ability and gel stabilizing ability and control the gel strength of the resulting gel.

When used in combination with agar, they can also mitigate water release and improve the texture of the resulting gel.

Each gelling agent and thickening agent is added to the gel composition of the invention typically in an amount ranging from about 0.05-0.3 wt. %.

Masking Agent

In the gel composition of the invention, a masking agent is preferably incorporated.

Examples of masking agents include fruit juice, fermented milk, hard-to-digest dextrin, hydrogenated resistant maltodextrin, nigerooligosaccharide and trehalose.

Among the above masking agents, fruit juice, hard-to-digest dextrin and hydrogenated resistant maltodextrin are particularly preferable.

The proportion of masking agent in the gel composition is preferably about 0.1 to about 20 wt. %, and more preferably about 0.5 to about 15 wt. %.

The addition of a masking agent provides the composition with a high nutritive value and excellent taste and flavor.

Vitamin D

Vitamin D is preferably incorporated in the gel composition to enhance calcium absorption. The combined use of calcium and vitamin D produces significant effects such as enhancement of calcium absorption in the intestinal tract and elevation of blood calcium concentration.

Vitamin D includes vitamin D₂ and vitamin D₃, which are different in side chain structure. Both forms of vitamin can be used as the vitamin D of the invention.

The proportion of vitamin D in the composition is preferably in the range of about 0.1×10⁶ to about 10×10⁻⁶ wt. %, and more preferably about 0.3×10⁻⁶ to about 5×10⁻⁶ wt. %.

Method of Producing the Gel Composition

The gel composition of the invention is prepared by mixing the specified amount of the components mentioned above with the specific amount of water with heating, emulsifying the mixture and then cooling the mixture. Such emulsification can be conducted by adding all the components to water at once and then carrying out a minor mechanical operation such as stirring. Alternatively, it can be conducted by preliminarily preparing an aqueous solution of the water-soluble components, adding to the solution the oil-soluble components and an emulsifying agent or a mixture thereof and subjecting them to a similar mechanical operation such as stirring. The latter is preferable to obtain a more uniformly emulsified mixture.

The above mixing operation (emulsification operation) can be performed at room temperature, but is preferably performed while heating at 30 to 60° C. The emulsifying operation can be conducted in a conventional manner with a suitable homogenizer, for example, homomixer, high-pressure homogenizer or the like, by complete passage process or circulation process.

For example, the following preferable method can be used to prepare the gel composition of the invention.

To a mixture (dispersion) of protein materials, citric acid and water, are added fats, emulsifying agents, carbohydrates, calcium materials, and other additive components. The obtained emulsion is heated to about 60° C. The emulsion is then mixed with a solution prepared by dissolving agar and other gelling agents or thickening agents by heating in water which had been previously heated to about 80° C.

Promoter for Increasing Plasma Volume

The promoter for increasing plasma volume of the invention contains the above gel composition as an active ingredient and induces plasma volume increasing effects that are concluded to be based on the synergetic effects of the intake of the specific components and exercise.

The promoter for increasing plasma volume of the invention is suitable, for example, for improving heat tolerance and preventing heat illness.

The promoter of the invention can be prepared by using the above gel composition as it is or mixed with a suitable carrier and formulating into a pharmaceutical preparation according to known methods.

The promoter of the invention is obtainable by cooling the gel composition, preferably by placing the gel composition in a suitable container, sterilizing and cooling the gel composition.

Suitable containers are any of those made of plastics and used as storage containers.

Examples of container materials include polyethylene, polypropylene, stretched polyamide, polyethylene terephthalate, Eval (ethylene vinyl alcohol copolymer resin, product of KURARAY CO.,LTD.) and composite materials produced by laminating these resins and aluminum, paper or the like. Examples of commercially available containers include Soft Pouch (trademark manufactured by FUJI SEAL, INC.), Bottled Pouch (trademark, manufactured by TOPPAN PRINTING CO., LTD.), Spouch (trademark, manufactured by DAI NIPPON PRINTING CO., LTD.) and Cheerpack (trademark, manufactured by HOSOKAWA YOKO CO., LTD.).

Sterilization can be performed in a conventional manner such as by heating. In this case, sterilization also serves as heating and thus makes preceding heating unnecessary.

Although the dose of the promoter of the invention is not specifically limited, it is preferable to administer the promoter in an amount of about 3.2 g per kg of body weight shortly after endurance exercise(5 to 10 minutes later) with regard to excellent plasma volume increase promotion.

Food

The promoter for increasing plasma volume of the invention can be used not only as a pharmaceutical preparation as mentioned above but also used by being contained in foods.

The food of the invention can be prepared by incorporating the promoter of the invention into a suitable foods or food materials.

The food of the invention can be used as a food to increase plasma volume, food for improving heat tolerance, and food for preventing heat stroke. It can also be used as a health drink, health food, food for specified health use, dietetic food, etc.

The proportion of promoter in the food can be suitably determined according to the use and purpose.

The food of the invention may, if desired, further contain appropriate additive substances in addition to the promoter.

Examples of such substances include sweeteners such as natural sweeteners (other than carbohydrates) and synthetic sweeteners, vitamins and minerals (electrolytes and trace elements), flavoring agents such as natural flavors, synthetic flavors, coloring agents, flavor enhancing substances (chocolate, etc.), food preservatives, natural fruit juices, and natural fruit fleshes.

Examples of natural (non-carbohydrate) sweeteners include thaumatin, stevia extract (rebaudioside A, etc.), glycyrrhizin, and the like.

Examples of synthetic sweeteners include saccharin, aspartame and the like.

Examples of vitamins include water-soluble and fat-soluble vitamins such as vitamin A (retinols), vitamin B₁ (thiamine), vitamin B₂ (riboflavin), vitamin B₆ (pyridoxine), vitamin B₁₂ (cyanocobalamin), vitamin E (tocopherol), niacin, bisbentiamine, nicotinamide, calcium pantothenate, folic acid, biotin, choline bitartrate and the like. These vitamins can be used as multivitamins containing various vitamins.

Examples of minerals (electrolytes and trace elements) include known minerals such as sodium chloride, sodium acetate, magnesium sulfate, magnesium chloride, dipotassium phosphate, monosodium phosphate, ferric citrate, ferrous pyrophosphate, ferric pyrophosphate, iron and sodium succinatocitrate, manganese sulfate, cupric sulfate, zinc sulfate, sodium iodide, potassium sorbate, zinc, manganese, copper, iodine, cobalt and the like.

Examples of flavoring agents include apple flavors, orange flavors, grapefruit flavors, lemon flavors, pineapple flavors and the like. These flavoring agents include natural and synthetic flavors.

Examples of coloring agents include Red No.2, Red No.3, Green No.3, Blue No.1, Blue No.2, Yellow No.4, Yellow No.5, red cabbage color, orange pigment, gardenia pigment, chlorophyll, perilla color, tomato pigment, safflower pigment and the like.

Examples of flavor enhancing substances include chocolate.

Examples of food preservatives include butyl hydroxyanisole (BHA), dibutylhydroxytoluene (BHT), sodium nitrate, sodium nitrite, disodium ethylenediaminetetra-acetate (EDTA), tert-butylhydroquinone (TBHQ), benzoic acid, Japanese styrax benzoin extract, rumput roman extract, hinokitiol extract, pectin digests, Magnolia obovata extract, forsythia extract and the like.

Examples of natural fruit juices and natural fruit fleshes include those of apples, green apples, oranges, mandarin oranges, grapefruits, peaches, strawberries, muscats, grapes, pineapples, lemons, pears, litchis, blueberries, mangos, bananas and like-fruits.

Among these, the addition of vitamins and minerals are desirable in view of nutritional support.

Examples of particularly preferable vitamins include a multivitamin (hereinafter referred to as “multivitamin 1”) of the following components:

| | | |
|---|---|---|
| Vitamin A | 10-2000 | IU |
| Vitamin B₁ | 0.01-3.0 | mg |
| Vitamin B₂ | 0.01-3.1 | mg |
| Vitamin B₆ | 0.01-3.2 | mg |
| Vitamin B₁₂ | 0.1-30 | μg |
| Vitamin E | 1-100 | IU |
| Nicotinamide | 0.1-30 | mg |
| Calcium pantothemate | 0.1-31 | mg |
| Folic acid | 0.01-3.0 | mg |

The above additive substances can be used singly or in combination of two or more.

The proportions of the substances in the gel composition are not particularly limited, but are usually used in such an amount that the total amount of additives is less than 2 parts by weight per 100 parts by weight of the gel composition. The promoter for increasing plasma volume and the food containing the promoter of the invention is obtainable by cooling, preferably by placing in a suitable container, sterilizing and cooling. Suitable containers are any of those made of plastics and used as storage containers. Examples of container materials include polyethylene, polypropylene, stretched polyamide, polyethylene terephthalate, Eval (ethylene vinyl alcohol copolymer resin, product of KURARAY CO.,LTD.) and composite materials produced by laminating these resins and aluminum, paper or the like. Examples of commercially available containers include Soft Pouch (manufactured by FUJI SEAL, INC.), Bottled Pouch (manufactured by TOPPAN PRINTING CO., LTD.), Spouch (manufactured by DAI NIPPON PRINTING CO.,LTD.) and Cheerpack (manufactured by HOSOKAWA YOKO CO., LTD.). Sterilization can be performed in a conventional manner such as by heating. In this case, sterilization also serves as heating and thus makes preceding heating unnecessary.

The following examples are provided to illustrate the present invention in further detail. In these examples, parts and percentages are all by weight unless otherwise specified.

### EXAMPLE 1

### Preparation of Promoter for Increasing Plasma Volume

To water were added the specified amounts of the components shown below and a suitable amount of other components, i.e., pineapple juice, multivitamin 1 and pineapple flavor, and mixed and stirred to form an emulsion, which was then heated to 80° C. 200 g of the emulsion was packed in a Spouch (manufactured by Dai Nippon Printing Co., Ltd.) and sterilized by heating at 80° C. for 10 minutes and cooled to provide a pouched gel beverage product containing the promoter in increasing plasma volume.

Protein: WPC (WPC-80) 4.0%, gelatin peptide 1.5%

Calcium: Milk calcium 0.4% (calcium content 140 mg %)

Acidulant: citric acid 0.5%, gluconic acid 0.3%, phosphoric acid 0.4%

Carbohydrate: sugar 10%, dextrin 2%

Fat: soybean oil 0.3%

Emulsifying agent: glycerine fatty acid ester 0.02%

Agar: 0.3%

Masking agent: fruit juice 1.0%, hydrogenated resistant maltodextrin 0.5%

Vitamin D: 3.7×10⁻⁶%.

To confirm the effect of the promoter in increasing plasma volume prepared in Example 1, the following evaluation was conducted.

I. Evaluation Method

(1) Subjects

Eight young persons (average age: 21.1±1.0) and eight elderly persons (average age: 68.1±1.7) were participated as subjects. Table 2 shows their age, body height and weight, BMI, and maximum oxygen uptake (VO₂max).

TABLE 2

| | | | Body weight | BMI | VO₂max |
|---|---|---|---|---|---|
| | Age (yrs) | Body height (cm) | (kg) | (kg/m²) | (ml/kg/min) |
| The young | 21.1 ± 1.0 | 170.7 ± 2.3 | 64.2 ± 3.4 | 21.9 ± 0.7 | 56.6 ± 1.4 |
| The elderly | 68.1 ± 1.7*** | 165.5 ± 2.1 | 64.0 ± 2.6 | 23.3 ± 0.6 | 36.5 ± 1.3*** |

Numerical values in the table denote Mean ± standard error. In Table 2,
***indicates a p < 0.001 for the young versus the elderly comparison.

As shown in Table 2, the age of the elderly is significantly higher than that of the young (p<0.001), and maximum oxygen uptake (VO₂max) of the young is significantly higher than that of the elderly (p<0.001).

(2) Exercise Load

Endurance exercise was performed on a bicycle ergometer for 72 minutes using an improvement of Nagashima's exercise load method (Nagashima K et al., Journal of Applied Physiology, January 2000; 88(1); p.41-46.). One set of exercise consisted of a 4-minute exercise at 80% VO₂ₚₑₐₖ intensity and a 5-minute exercise at 20% VO₂ₚₑₐₖ intensity. Eight sets of exercise were performed in total.

(3) Experimental Conditions

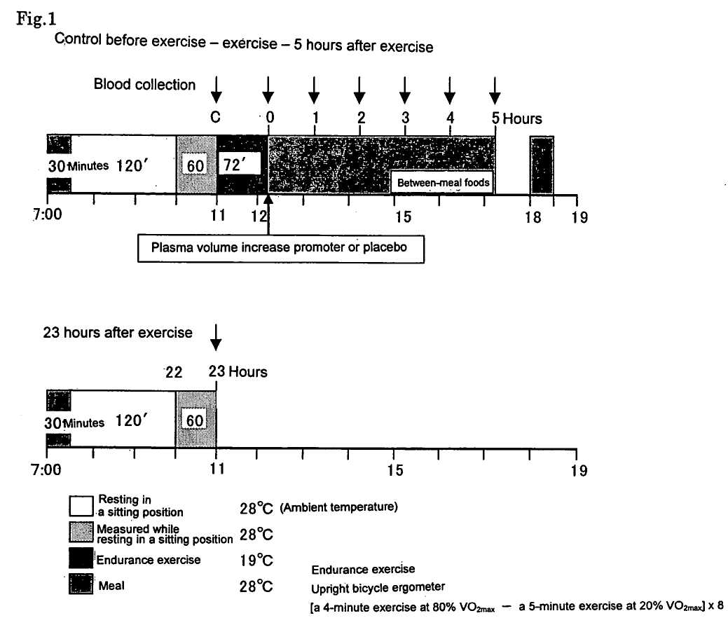

The experiment was carried out by administering the promoter for increasing plasma volume prepared in Example 1 or a placebo shortly after (5 to 10 minutes later) completion of the exercise load. The detailed experiment process is shown in FIG. 1.

The promoter administration and the placebo administration were performed with an interval of at least 1 week's washout period by the random crossover method. In other words, the experiment was carried out by administering the promoter and the placebo in a random order.

(4) Diet Control

From the dinner of the previous evening before exercise to the breakfast of the day following the exercise were set as standardized meals. The subjects ate standardized meals at specified times of the day. In addition, the subjects ate between-meal foods 16 times in total every 10 minutes from 2.5 hours after completion of the exercise. Table 3 shows the total energy and protein content of the standardized meals, between-meal foods, promoter for increasing plasma volume and placebo.

TABLE 3

| | The young | | The elderly | |
|---|---|---|---|---|
| | Energy | Protein | Energy | Protein |
| | intake | intake | intake | intake |
| | (kcal/kg) | (g/kg) | (kcal/kg) | (g/kg) |
| Dinner | 23.00 | 0.67 | 20.30 | 0.67 |
| Breakfast | 8.00 | 0.20 | 7.20 | 0.20 |
| Between-meal food | 4.00 | 0.14 | 4.00 | 0.14 |
| Promoter for | 3.08 | 0.18 | 3.08 | 0.18 |
| increasing | | | | |
| plasma volume | | | | |
| Placebo | 0.14 | 0.00 | 0.14 | 0.00 |

II. Evaluation Items

Blood samples were collected from the subjects 8 times in total, i.e., shortly before and shortly after the exercise load, every hour from 1 to 5 hours after exercise load, and 23 hours after exercise load, which was on the following day. After the blood samples were centrifuged, plasma volume (PV), plasma albumin content (Alb content), and plasma total protein content (TP content) were determined.

Statistical analysis was conducted with ANOVA for repeated measures which make comparisons between the two groups of subjects (the young versus the elderly) and the two test compounds (promoter administration versus placebo administration).

When the significance level was less than 5% (P<0.05), post hoc test (Scheffe's test) was performed to a difference at each points of time course.

III. Evaluation Results

(1) Plasma Volume

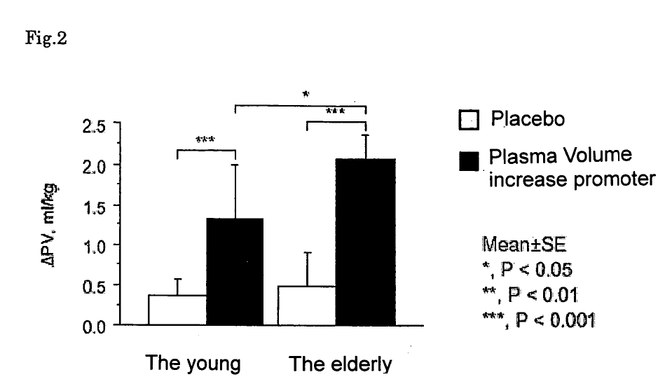

FIG. 2 shows the amount of change in plasma volume (PV), comparing before exercise (baseline set as 0, point C in FIG. 1: the first blood collection) and 23 hours after completion of the exercise (the eighth blood collection).

As shown in FIG. 2, promoter administration shortly after exercise significantly increases plasma volume both in the young and the elderly as compared with placebo administration (p<0.001). Compared to the young, the elderly showed the greater change in plasma volume with promoter administration (p<0.05).

(2) Total Plasma Protein Content

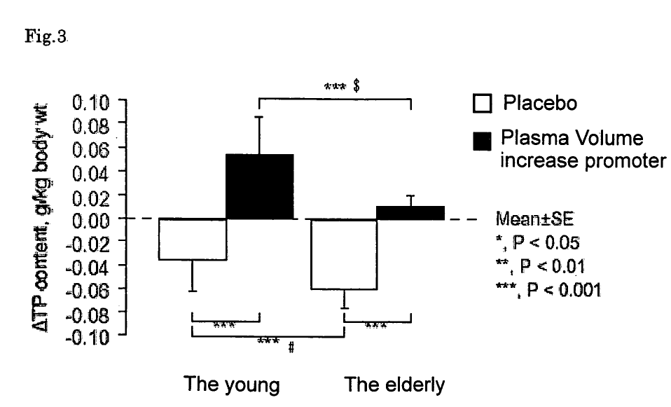

FIG. 3 shows the amount of change in plasma total protein content, comparing the content before exercise (baseline set as 0, point C in FIG. 1: the first blood collection) and the content 23 hours after completion of the exercise (the eighth blood collection).

As shown in FIG. 3, promoter administration shortly after exercise significantly increased plasma total protein both in the young and the elderly as compared with placebo administration (p<0.001). Compared to the elderly, the young showed the greater increase in plasma total protein content with promoter administration (p<0.001). Compared to the young, the elderly showed the greater reduction in plasma total protein content with placebo administration (p<0.001).

(3) Plasma Albumin Content

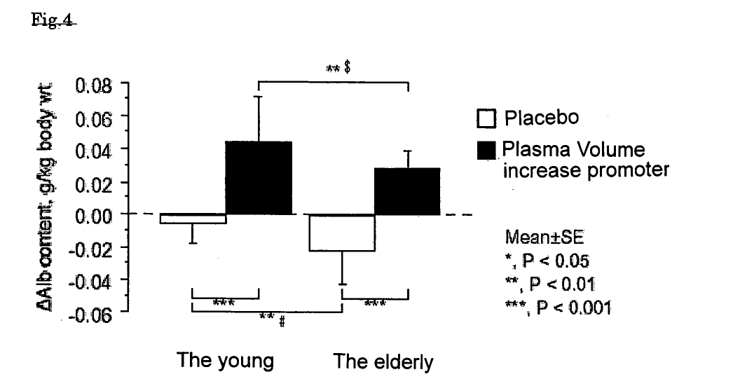

FIG. 4 shows the amount of change in plasma albumin content, comparing the content before exercise (baseline set as 0, point C in FIG. 1: the first blood collection) and the content 23 hours after completion of the exercise (the eighth blood collection).

As shown in FIG. 4, promoter administration significantly increased plasma albumin content both in the young and the elderly as compared with placebo administration (p<0.001). Compared to the elderly, the young showed the greater increase in plasma albumin content with promoter administration (p<0.01). Compared to the young, the elderly showed the greater decrease in plasma albumin content with placebo administration (p<0.01)

The evaluation results show clearly the following:

When the promoter for increasing plasma volume of the invention is administered to the elderly shortly after exercise, there is an increase in plasma volume as well as an increase in plasma total protein content and plasma albumin content.

In the young also, promoter administration shortly after exercise increases plasma volume as well as plasma total protein content and plasma albumin content, compared to placebo administration.

1. A promoter for increasing plasma volume containing as an active ingredient a gel composition comprising the following components and having a pH in the range of 3 to 4:

| | | |
|---|---|---|
| Protein that does not coagulate at | 3-8 | wt. % |
| pH 3 to pH 4 | | |
| Calcium | 0.1-0.5 | wt. % |
| Acids | 0.5-3 | wt. % |
| Carbohydrate | 4-20 | wt. % |
| Fat | 0-5 | wt. % |
| Emulsifying agent | 0-0.5 | wt. % |
| Agar | 0.1-1 | wt. % |
| Water | 65-90 | wt. % |

2. The promoter according to claim 1 wherein the gel composition further comprises 0.1 to 20 wt. % of at least one masking agent selected from the group consisting of fruit juice, fermented milk, hard-to-digest dextrin, hydrogenated resistant maltodextrin, nigerooligosaccharide and trehalose.

3. The promoter according to claim 1 or 2 wherein the gel composition further comprises vitamin D in an amount of 0.1×10⁻⁶ to 10×10⁻⁶ wt. %.

4. A food containing the promoter of any of claims 1 to 3.
